(19) United States
(12) Patent Application Publication
(10) Pub. No.: US 2012/0010727 A1
(43) Pub. Date: Jan. 12, 2012

(54) AMNION AND CHORION CONSTRUCTS AND USES THEREOF IN SPORT INJURY SURGERIES

(75) Inventors: Robin R. YOUNG, Wayne, PA (US); Richard M. JAY, Philadelphia, PA (US)

(73) Assignee: AFcell Medical, Parsippany, NJ (US)

(21) Appl. No.: 13/177,177

(22) Filed: Jul. 6, 2011

Related U.S. Application Data
(60) Provisional application No. 61/361,563, filed on Jul. 6, 2010.

(51) Int. Cl. A61F 2/02

(52) U.S. Cl. 623/23.72

(57) ABSTRACT

Improved methods for sport injury surgeries are described. The improvement includes covering a damaged site of fascia with at least one of an amniotic fluid and a construct for use in surgical repair of the sport injury prior to wound closing. The construct contains an allograft comprising at least one layer of human amnion and chorion tissues and the construct has a size and shape suitable for covering the damaged site of fascia. The method improves fascial membrane repair, reduces complications and recovery time of sport injury surgeries.

### CROSS-REFERENCE TO RELATED APPLICATIONS

This application is entitled to priority pursuant to 35 U.S.C. §119(e) to U.S. Provisional Patent Application No. 61/361,563, filed Jul. 6, 2010 which is hereby incorporated by reference herein in its entirety.

### BACKGROUND OF THE INVENTION

The human fascia support structure is a thin, elastic membrane network that exists in continuous layers throughout the body. More specifically, the human fascia surrounds all human internal body parts, muscles, tendons, ligaments and bones. During gestation and in the development stage of the mesoderm, bone, muscles, nerves, tendons and fascial membranes develop as one basic tissue with varying degrees of elasticity, stability and mutability. Fascial membranes are the most elastic and change most extensively when injured. The human body relies extensively on fascial membranes to position, tone, cover, lubricate, protect and allow articulating hard and soft tissues (e.g. muscles, and tendons) to glide freely. Therefore, fascial membranes are critical to the body's ability to move athletically. The following are examples of fascial membranes: superficial fascia (i.e., hyperdermis), which is between the skin and muscles, houses much of the body fat, stretches and adjusts to strains of all kinds and allows an athlete's muscles to glide so well under the skin; periosteum, which covers each bone; perichondrium, which covers cartilage; synovial, which lines joint cavities; deep fascia, which is a denser material that covers each skeletal muscle and permits structures to glide and slide over each other, such as dural membrane and meninges that cover the brain and spinal cord.

Sport injuries, i.e., injuries that occur to people, particularly athletes, training for or participating in sporting events, invariably disrupt one or more of the fascial membranes. When such a disruption occurs, whether from injury or in the process of surgical or non-surgical repair, the patient will usually experience increased pain, reduced mobility, less lubrication between articulating tissues (e.g. bone on bone grinding or tethering of muscles or nerves with adjacent tissues), less shock absorption and the risk of post operative scarring or adhesions. Such injuries range from bruises and muscle strains, to fractures, torn ligaments or tendons, and head injuries.

When the fascial membranes are injured, stressed or traumatized, for example during sport injuries, the fascial membranes respond by increasing tensional forces thus efficiently making a “sling” over the injured bones, muscles or nerves. The membranes also respond to trauma by “gluing” affected areas of a site of injury. After the injury has healed, the fascial membranes often “forget” to un-glue and athletes then experience a layer of tension and adhesion at the injury site. In those cases, the protective fascial membranes no longer slide, causing adjacent structures to tether or tug at each other. Muscles, bones, tendons, nerves are all subject to such post-trauma effects due to injuries to fascial membranes.

It's difficult to return to competitive athletics after any serious injury. Surgeries have been used to treat sport injuries. For example, Anterior Cruciate Ligament (ACL) reconstruction surgery has been used to help competitive athletes to return to competition after ACL tearing. During surgery the old ACL is removed, a graft from the patellar tendon or the hamstring is prepared, holes are drilled in the tibia and femur and the graft is attached with screws to the bones. Often 6 to 9 months of rehabilitation after the surgery is required to strengthen the areas surrounding the graft so that it does not fail. One of the reasons that athletes undergo such extensive rehabilitation exercises is to readjust the tensional dynamics of the fascial membrane network and allow muscles, tendons, ligaments, bones and even internal organs to move back into proper alignment.

Issues related to sport injuries are not trivial and affect, we estimate, in excess of 30 million sport medicine patients annually. There is a need to improve the treatment and recovery from sport injuries, particularly to improve the repair of injured fascial membranes.

During gestation of an embryo, the human body creates its largest single sheet of fascial membrane—the amnion. The amnion is a thin, cellular, extraembryonic membrane that forms the inner membrane of a closed sac surrounding and protecting an embryo in reptiles, birds, and mammals. The sac contains the fetus and amniotic fluid or liquor amnii, in which the embryo is immersed, nourished and protected. Typically, the amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid. The amnion has multiple functions, e.g., as a covering epithelium, as an active secretary epithelium, and for intense intercellular and transcellular transport.

Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries as documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and tendon repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograft for repairing corneal defects. See, for example, Tsai and Tseng, Cornea. 1994 September; 13(5):389-400; and Dua et al., Br. J. Ophthalmol 1999, 83:748-752. In addition, amnion and amniotic fluid have recently been used as sources of placental stem cells. See, e.g., U.S. Pat. No. 7,255,879 and WO 200073421.

Despite the clinical and published record regarding the safety and efficacy of amnion in broad surgical use, issues regarding reproducibility, safety and the precise form of amnion for each prospective indication have prevented amnion from achieving broad commercial distribution.

It has now been discovered that using allograft patches of various sizes and shapes comprising amnion as well as other birth tissue products such as chorion and amniotic fluid in sport injury surgeries, significantly reduces inflammation and tissue adhesion, promotes uniform re-growth and epithelialization, and prevents scar tissue formation, thus significantly improving fascial membrane repair and reducing complications and recovery time from sport injury surgeries.

### BRIEF SUMMARY OF THE INVENTION

In another general aspect, the present invention relates to a construct for use in surgical repair of a sport injury, the construct comprising an allograft comprising at least one layer of human amnion and chorion tissues, wherein the construct has a size and shape suitable for covering a damaged site of fascia.

In another general aspect, the present invention relates to a method of performing a surgical repair of a sport injury in a subject, comprising:
a. surgically repairing the sport injury in the subject; and
b. covering a damaged site of fascia with at least one of an amniotic fluid and a construct for use in surgical repair of the sport injury prior to wound closing,

wherein the damaged site of fascia results from at least one of the sport injury and the surgical repairing step, and the construct comprises an allograft comprising at least one layer of human amnion and chorion tissues, wherein the construct has a size and shape suitable for covering the damaged site of fascia.

Yet another general aspect of the present invention relates to a kit, comprising:
a. at least one of an amniotic fluid and a construct for use in surgical repair of a sport injury; and
b. instructions on how to use the amniotic fluid and the construct in the surgical repair,

wherein the construct comprises an allograft comprising at least one layer of human amnion and chorion tissues, the construct has a size and shape suitable for covering a damaged site of fascia.

In a preferred embodiment of the present invention, the amniotic fluid and the human amnion and chorion tissues used in the present invention are obtained by a process comprising:
a. obtaining informed consent from pregnant females;
b. conducting risk assessment on the consented pregnant females to select an amnion donor;
c. procuring the amniotic fluid from the amnion donor;
d. procuring after birth placenta from the amnion donor; and
e. obtaining the human amnion and chorion tissues from the placenta.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 are schematic diagrams that illustrate some constructs of various sizes of double wing shapes according to embodiments of the present invention;

FIG. 2 are schematic diagrams that illustrate some constructs of various sizes of single wing shapes according to embodiments of the present invention;

FIG. 3 are schematic diagrams that illustrate some constructs of various sizes of burn covers shapes according to embodiments of the present invention;

FIG. 4 are schematic diagrams that illustrate some constructs of various sizes of diamond shapes according to embodiments of the present invention;

FIG. 5 are schematic diagrams that illustrate some constructs of various sizes of articulating cup shapes according to embodiments of the present invention;

FIG. 6 are schematic diagrams that illustrate some constructs of various sizes of ligament, tendon or nerve tube shapes according to embodiments of the present invention; and

FIG. 7 is a schematic diagram that illustrates the contents of a kit comprising a plurality of constructs for use in sport injury surgery according to an embodiment of the present invention.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

According to embodiments of the present invention, amniotic fluid, amnion and/or chorion tissues are used to improve sport injury surgeries.

In one general aspect, embodiments of the present invention relate to a method of performing a surgical repair of a sport injury in a subject, comprising:
(a) surgically repairing the sport injury in the subject; and
(b) covering a damaged site of fascia with at least one of an amniotic fluid and a construct for use in surgical repair of the sport injury prior to wound closing,

wherein the damaged site of fascia results from at least one of the sport injury and the surgical repairing step, and the construct comprises an allograft comprising at least one layer of human amnion and chorion tissues, and the construct has a size and shape suitable for covering the damaged site of fascia.

The amniotic fluid and the construct for use in surgical repair of the sport injury can be applied to the damaged site of fascia individually or in combination. Preferably, the amniotic fluid is processed so that it has a relatively high viscosity for ease of application and for remaining in the desired area after the application.

In an embodiment of the present invention, both the amniotic fluid and the construct for use in surgical repair of the sport injury are applied to the damaged site of fascia resulting from the sport injury or the sport injury surgery.

In another embodiment of the present invention, only the amniotic fluid is applied to a damaged site of fascia, preferably the amniotic fluid has a relatively high viscosity.

In a preferred embodiment of the present invention, a construct for use in surgical repair of the sport injury according to an embodiment of the present invention is applied over a damaged site of fascia during a surgical repair of a sport injury, preferably after the sport injury is sutured or repaired.

Methods according to embodiments of the present invention improve the ability of soft tissues to heal rapidly from sport injuries including, but not limited to, anterior cruciate ligament (ACL) injuries, Achilles tendon ruptures, frozen shoulder (adhesive capsulitis), ankle fractures and injuries, anterior and posterior cruciate ligament injury, chronic blister treatment, bursitis, carpal tunnel, cartilage injuries, elbow injuries, finger fractures, fractured clavicles, golfer's elbow (medial epicondylitis), torn groin muscle, hamstring tears, hand ischemia, hip pointer, patellofemoral pain syndrome, lateral epicondylitis (tennis elbow), medial and lateral collateral ligament injury, plantar fasciitis, quadricep pulls and tears, rotator cuff tears, shoulder tendinitis and impingement.

For all of these indications, the allograft and/or amniotic fluid covers, protects and restores the lost functionality of the fascial membranes at the site of injury or surgery.

Amnion or amniotic fluid completely lacks of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion has a broad spectrum of antimicrobial activity against bacteria, fungi, protozoa, and viruses for reduced risk of post-operative infection.

As a result, for all of these indications, the allograft and/or amniotic fluid effectively reduces post operational pain and risk of adhesion, and allows the athlete to experience a shorter but more successful rehabilitation period.

In one embodiment of the present invention, the allograft and/or amniotic fluid contains antimicrobial compounds with broad spectrum activity against bacteria, fungi, protozoa, and viruses, which reduce risk of post-operative infection.

Methods of the present invention apply to both open surgical procedures and minimally invasive surgical procedures such as arthroscopic, endoscopic or catheter based procedures.

The open surgical procedures involve an actual incision that opens the joint in order for repair or reconstruction of the injured structures. Open procedures are more invasive than arthroscopic procedures because the joint is exposed. Examples of open surgical procedures include, but are not limited to, rotator cuff repairs, ulnar collateral ligament reconstructions, ankle reconstructions, shoulder reconstructions, and fracture repairs.

According to an embodiment of the present invention, during an open sport injury surgical procedure an allograft of a pre-made suitable size and shape and/or amniotic fluid is applied to cover a damaged site of fascia resulting from the sport injury and/or surgery procedure.

Arthroscopy is a less invasive sports surgery procedure, utilizing a minimal incision, a cannula, small retractors, camera and/or small tools to work inside of a joint. It usually involves 3-4 small portal incisions into the joint, rather than one large incision. The exposure of the actual joint is minimized. While performing the procedure the surgeon relies on such external tools as cameras or view screens to augment visualization of the surgical site. Arthroscopic procedures have become very common, and are often done as an outpatient procedure. Examples of arthroscopy include, but are not limited to, meniscus repairs and meniscectomies, articular cartilage surgery, patella lateral release, labral repairs, and some rotator cuff repairs.

According to an embodiment of the present invention, during an arthroscopy sport injury surgery, an amnion allograft of a size and shape and/or an amniotic fluid is applied to cover a damaged site of fascia resulting from the sport injury.

For illustration purpose, a method according to an embodiment of the present invention is applied to a meniscectomy or meniscus repair, one of the most common types of sports injury surgery.

A tear of a meniscus is a rupturing of one or more of the fibrocartilage strips, i.e., menisci, in the knee. Meniscus tears may cause, for example, knee pain and swelling, and joint locking, when the patient is unable to fully straighten the leg, which can be accompanied by a clicking feeling. Meniscal tears may also cause a sensation that the knee gives away.

Some meniscus tears are not very good at healing on their own, surgery is often needed. The meniscus tear is first found using methods known in the art, such as arthroscopic evaluation. After the meniscus tear is found, the surgeon determines to either remove it, or repair it. This decision is based on where the tear is located within the meniscus. Tears located more toward the middle of the meniscus are removed, while tears toward the outside can sometimes be repaired.

During a meniscectomy, a small shaver is used to remove the torn part of the meniscus. Only the torn sections are removed to save as much of the meniscus as possible. Once the torn section is removed, an allograft patch comprising at least one layer of amnion and chorion tissues having a size and shape matching the exposed cross section of meniscus is used and placed over the exposed cross section of meniscus to thereby cover the damaged site of fascia resulting from the meniscectomy. The tools and arthroscope are removed, and the portal incisions are closed with either sutures or staples.

According to an embodiment of the present invention, an amniotic fluid is applied to the meniscus, before and after the torn section is removed, to thereby cover the damaged site of fascia resulting from meniscectomy, alone or in combination with the allograft patch comprising at least one layer of amnion and chorion tissues.

According to an embodiment of the present invention, one or more allografts comprising at least one layer of amnion and chorion tissues is placed over one or more suture lines and incisions resulting from the meniscectomy to form a cover and barrier over the suture lines and the incisions.

In an embodiment of the present invention, a plurality of allograft patches, each having a shape and size suitable for a particular damaged site of fascia, are used to cover a plurality of damaged sites of fascia resulting from a sport injury surgery.

In an embodiment of the present invention, the allograft to be used to cover a damaged site of fascia is able to be attached or affixed with fibrin glue, be able to adhere by using such other surgical glues as the amnion glutaraldehyde combination material commonly known as BioGlue®, or hold a 3.0 or 4.0 absorbable suture.

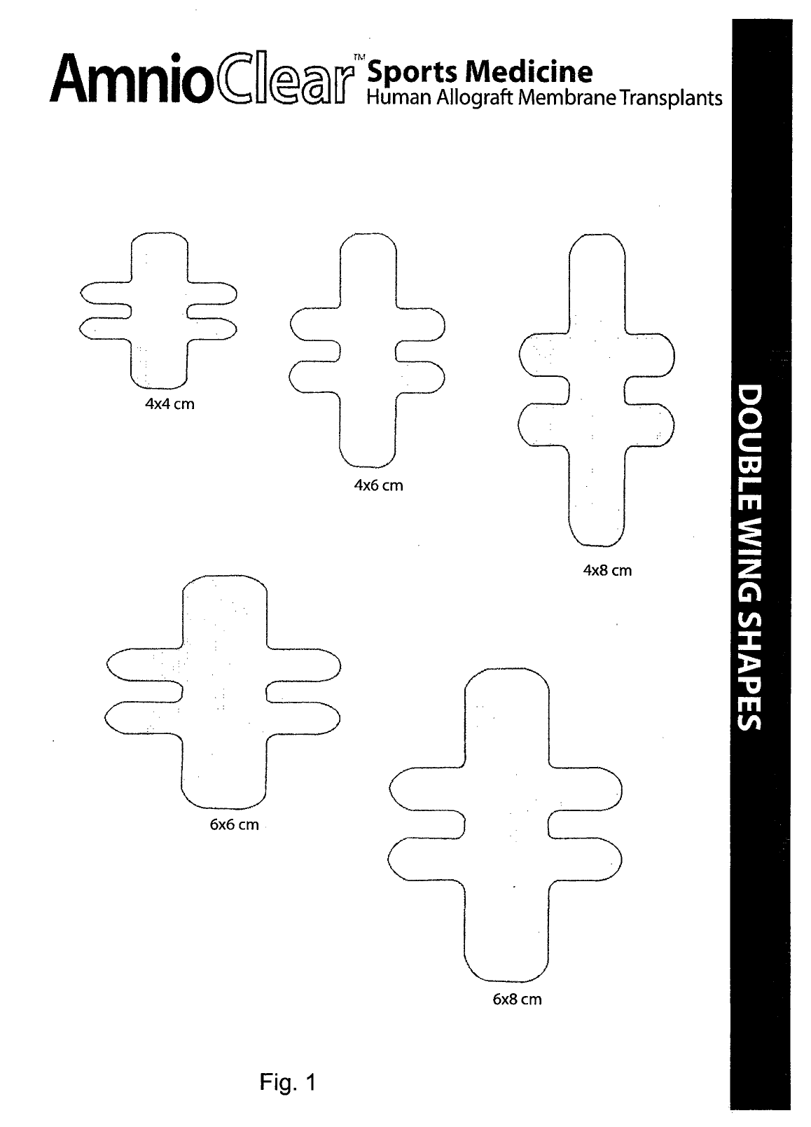

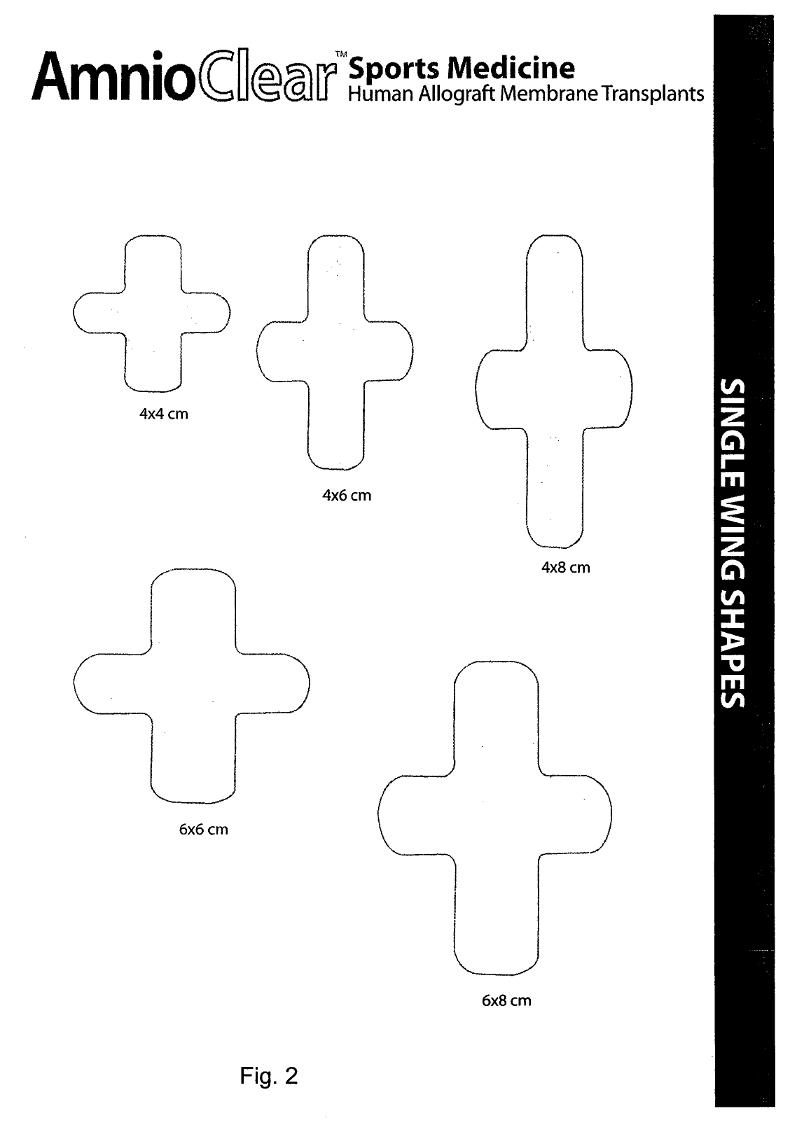

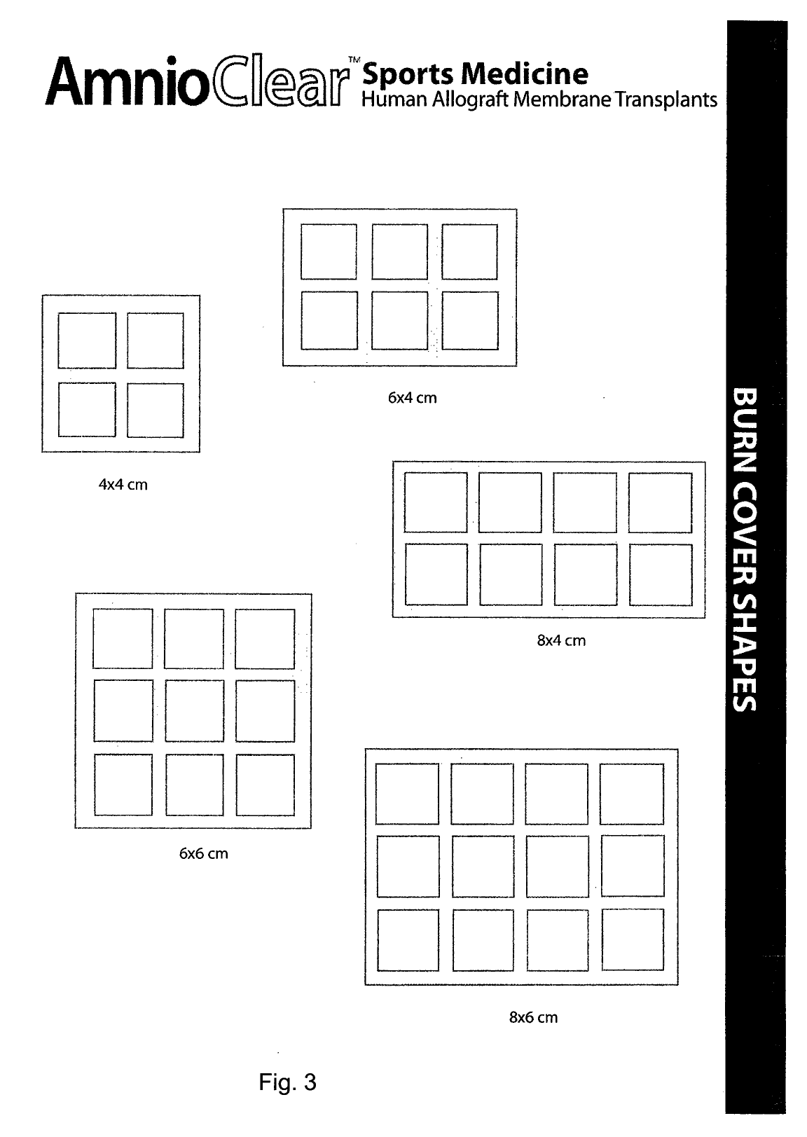

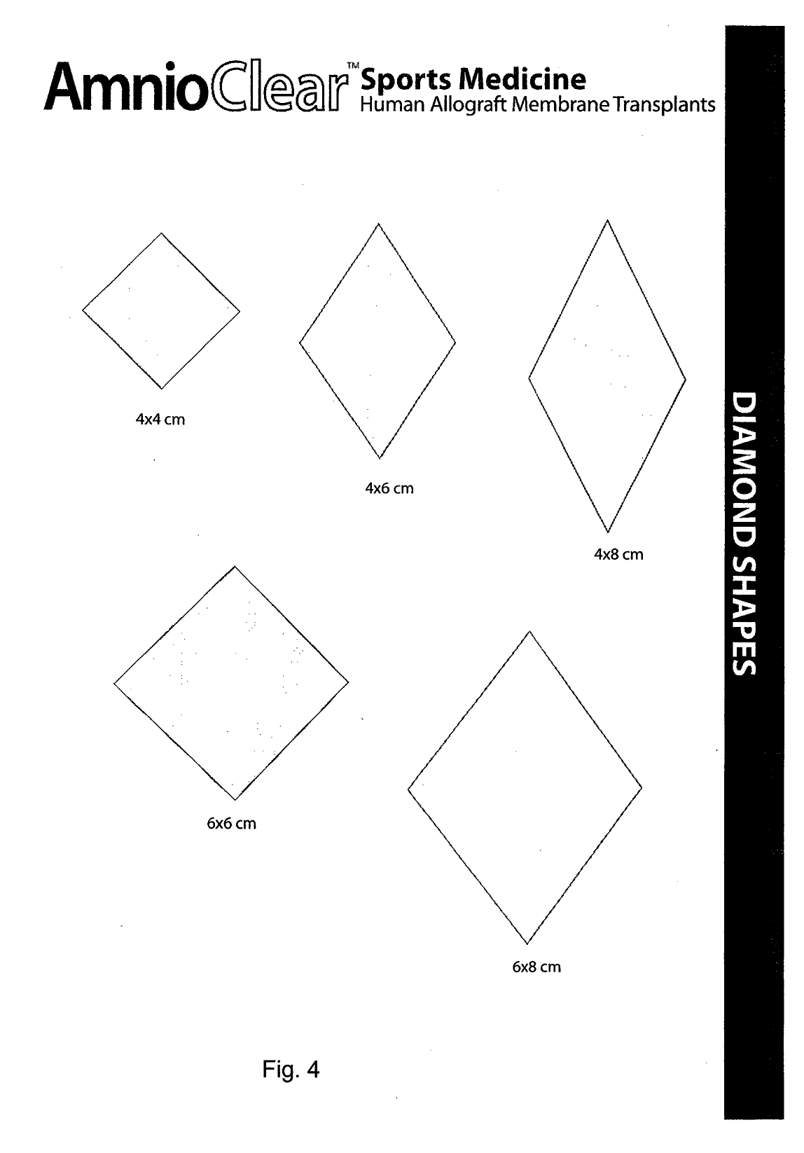

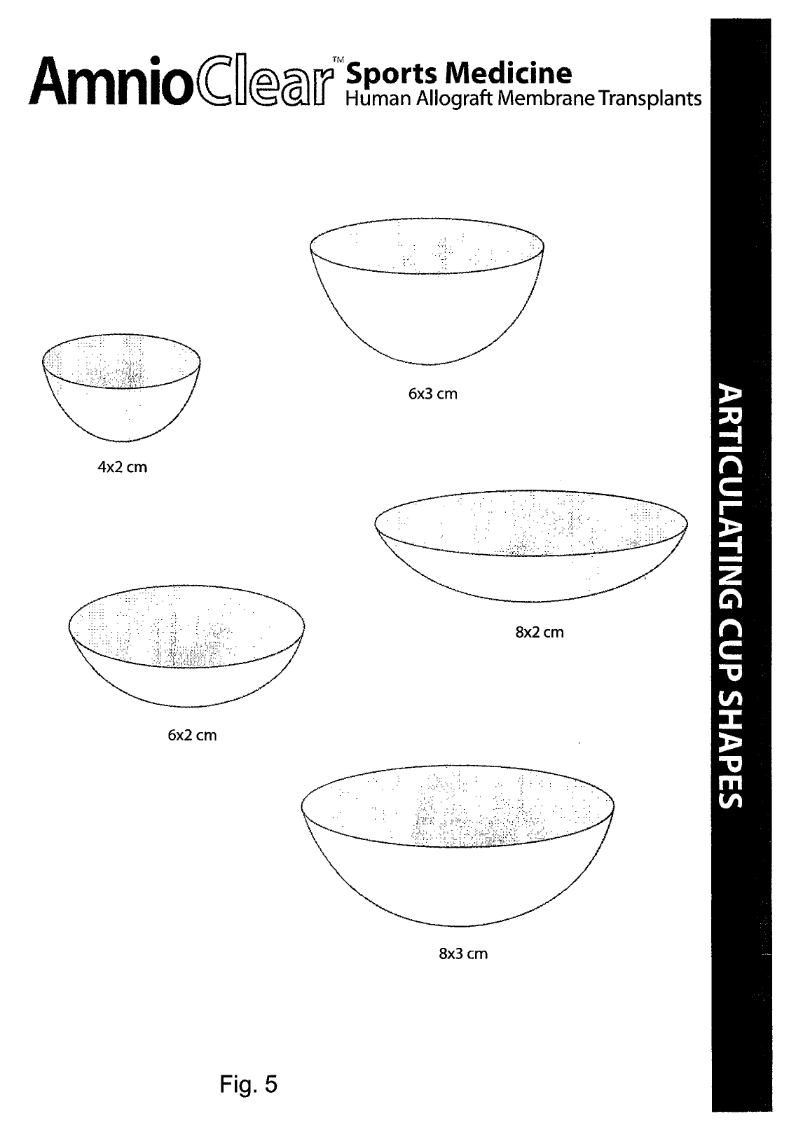

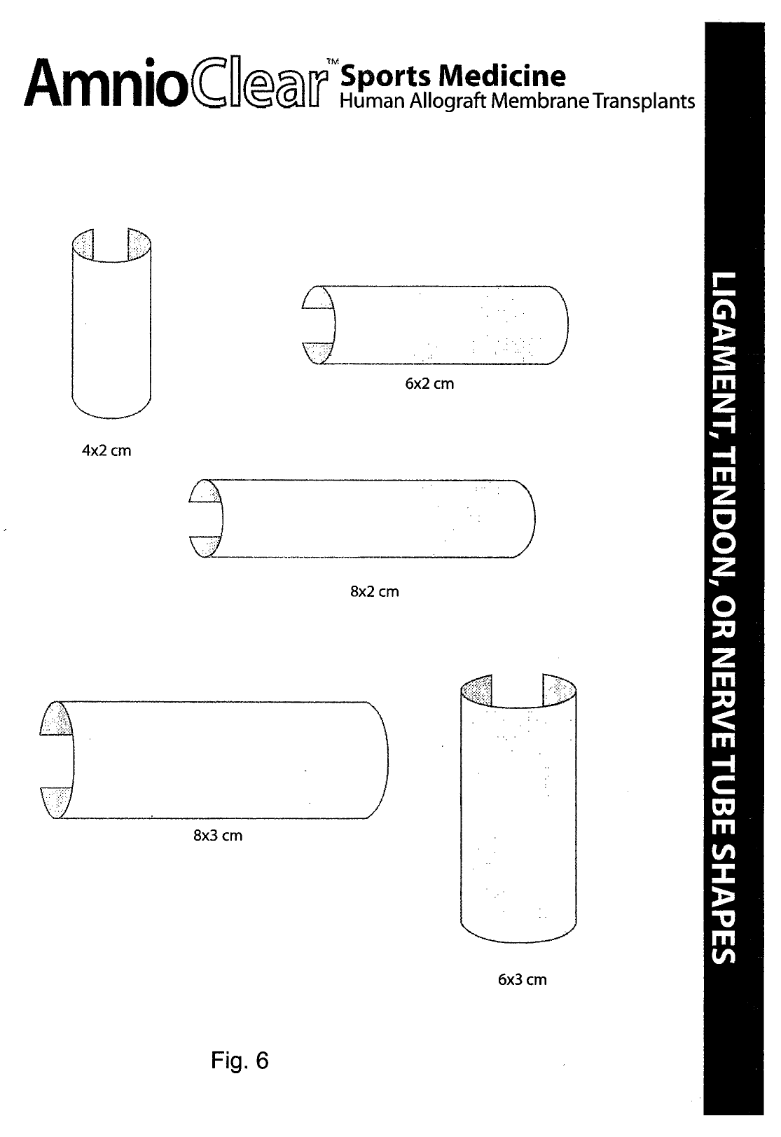

The appropriate shape and dimension of the allograft patch are chosen based on the shape and size of the damaged site of fascia. For example, the allograft patches can have the double wing shapes, single wing shapes, burn cover shapes, diamond shapes, articulating cup shapes, ligament, tendon or nerve tube shapes, at various sizes as those illustrated in FIGS. 1-6, respectively.

Preferably, the allograft patch is thin. In one embodiment of the invention, the allograft patch has a thickness of about 0.02 mm to 0.10 mm. It can have a single layer of amnion or chorion, more than one layers of amnion or chorion, a combination of one or more layers of amnion and one or more layers of chorion, or a combination of one or more layers of amnion and one or more layers of other collagen membranes of biological origin. The multiple layers in the allograft can be subject to a cross-linking treatment to make the layers closely adhere to each other in an integrated form.

In one embodiment of the present invention, the construct further comprises a frame, which can be flexible, semi-rigid or rigid, preferably a rigid or semi rigid frame. The thickness of the frame can be between 0.5 mm to 2 mm and the length and circumference are the same as the allograft tissue(s) bonded to it. In one embodiment, the frame is disposable. In another embodiment, the frame is implantable and resorbable. When a frame is used, in the case of either dry, wet or frozen allograft tissues, it facilitates the allograft tissues to be implanted over the damaged site of fascia.

In one embodiment of the present invention, an allograft comprising at least one layer of human amnion and chorion tissues is dried into a flat sheet, with or without a frame, and the dried allograft is used as the construct for the surgeries.

In an embodiment of the present invention, when a disposable frame is used, the dried tissue retains the shape of the frame when removed from the frame or could be packaged and sterilized with a disposable frame to retain its shape prior to use. The disposable frame can be removed and discarded prior to the use of the tissue. The disposable frame can be longer than the tissue for ease of handling and removal.

In another embodiment of the present invention, the allograft in the replacement cover is reinforced with an implantable and resorbable rigid or semi rigid polymer frame of a shape appropriate for covering a damaged site of fascia. This implantable and resorbable frame could be a mesh or a solid frame with several holes throughout.

The allograft, such as human allograft comprising one or more layers of amnion and/or chorion tissues, is bonded to the frame by various methods in view of the present disclosure, such as, drying the tissue on the frame, using a resorbable adhesive, keeping the tissue wet and laying it on the frame, or freezing the tissue on the frame.

In one embodiment of the present invention, one or more corners of the construct or allograft are rounded or flatted to prevent the corners from catching during implantation. In view of the present disclosure, any method known to those skilled in the art can be used to make the corners of the construct or allograft round or flatten.

In one embodiment of the present invention, the construct can carry one or more therapeutic agents, such as morphogenic proteins, small molecule compounds, pharmaceutical agents, anti-microbial agents, anti-inflammatory agent, agents that prevent scarring, adhesions and tethering of internal tissue of the sport injury site or the surgery site, analgesics, etc., to further improve the performance and reduce the complications of sport injury surgeries. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

The two surfaces of human amnion are structurally different. The surface facing the fetus is smooth and hardly cell adhesive, comprising a thin layer of fine fibers. The surface facing the chorion is rough and suitable for cell proliferation, comprising thick fasciculus. In one embodiment of the present invention, the allograft patch is placed adjacent to the damaged site of fascia so that the chorion facing surface of the amnion faces the fascia. In another embodiment of the present invention, the allograft patch is placed adjacent to the damaged site of fascia so that the fetus facing surface of the amnion faces the fascia. The surgeon is provided with a range of sizes, thicknesses and shapes of allograft patches, such as the diamond shape, the articulating cup shape, etc., which can be chosen and oriented according to the size and shape of the patient's anatomy.

In another embodiment of the present invention, a construct comprising at least one layer of amnion and chorion tissues is used to cover a skin incision resulting from the sport injury surgery. The allograft patch can be of any size suitable for covering the sutures or other type of tissue injuries at skin incision.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion.

Amnion membranes and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses. The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4 to 28° C., for example, in an ice bucket.

According to an embodiment of the invention, shortly after its expulsion after birth, a suitable human placenta is placed in a sterile zip-lock plastic bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results of screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation.

The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The prepared amnion and/or chorion can be sized into various shapes and sizes anticipated to be useful in sport injury surgery, such as those illustrated in FIGS. 1-6.

The present invention encompasses a kit, comprising
a. at least one of an amniotic fluid and a construct for use in surgical repair of a sport injury; and
b. instructions on how to use the amniotic fluid and the construct in the surgical repair,

wherein the construct comprises an allograft comprising at least one layer of human amnion and chorion tissues, the construct has a size and shape suitable for covering a damaged site of fascia.

In a preferred embodiment, the kit comprises a plurality of constructs for use in surgical repair of a sport injury. The construct can comprise one or more layers of amnion in combination with one or more layers of chorion or other collagen membranes of biological origin. Each of the plurality of constructs has a size and shape suitable for covering a damaged site of fascia resulting from a sport injury or a sport injury surgery. At least two of the plurality of constructs have different shapes or sizes in a kit. The construct can further comprise one or more therapeutically active agents, such as anti-microbial agents, growth enhancing agents, anti-inflammatory agents, analgesics, etc.

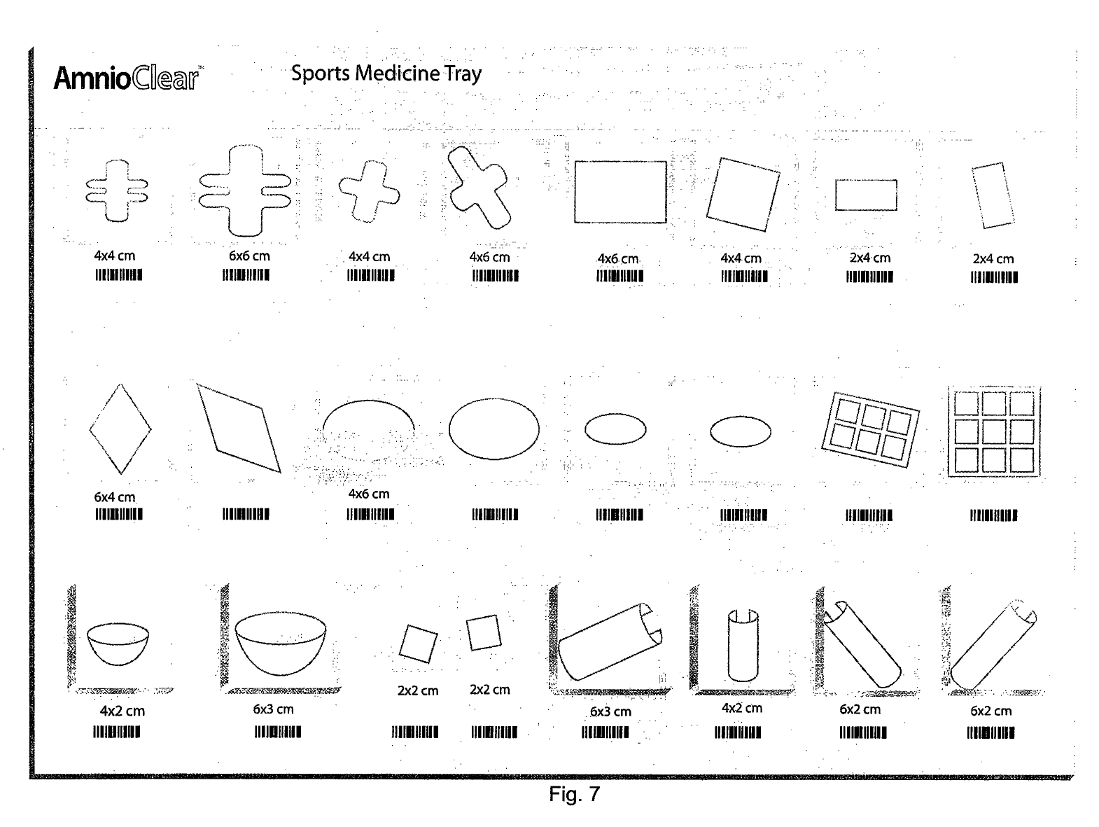

FIG. 7 illustrates a kit for sport injury surgery according to an embodiment of the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A construct for use in surgical repair of a sport injury, the construct comprising an allograft comprising at least one layer of human amnion and chorion tissues, wherein the construct has a size and shape suitable for covering a damaged site of fascia.

2. The construct of claim 1 further comprising a frame that is disposable or implantable and resorbable.

3. The construct of claim 2, wherein the frame is rigid or semi rigid.

4. The construct of claim 1, having a shape selected from the group consisting of double wing shapes, single wing shapes, burn cover shapes, diamond shapes, articulating cup shapes, ligament, tendon and nerve tube shapes.

5. The construct of claim 1, having one or more rounded or flattened corners.

6. The construct of claim 1, further comprising one or more active agents for treatment of the sport injury.

7. The construct of claim 6, wherein the one or more active agents are selected from the group consisting of morphogenic proteins, small molecule compounds, pharmaceutical agents, anti-microbial agents, anti-inflammatory agent, agents that prevent scarring, adhesions and tethering of internal tissue of the sport injury site or the surgery site, and analgesics.

8. The construct of claim 1, wherein the human amnion and chorion tissues are obtained using a process comprising:
a. obtaining informed consent from pregnant females;
b. conducting risk assessment on the consented pregnant females to select an amnion donor;
c. procuring after birth placenta from the amnion donor; and
d. obtaining the human amnion and chorion tissues from the placenta.

9. A method of preparing a construct of claim 2, the method comprising drying the allograft comprising at least one layer of human amnion and chorion tissues on the frame.

10. A method of performing a surgical repair of a sport injury in a subject, comprising:
a. surgically repairing the sport injury in the subject; and
b. covering a damaged site of fascia with at least one of an amniotic fluid and a construct for use in surgical repair of the sport injury prior to wound closing,
wherein the damaged site of fascia results from at least one of the sport injury and the surgical repairing step, the construct comprises an allograft comprising at least one layer of human amnion and chorion tissues and the construct has a size and shape suitable for covering the damaged site of fascia.

11. The method of claim 10, further comprising applying one or more allografts comprising at least one layer of human amnion and chorion tissues over one or more suture lines and incisions resulting from the surgical repair to form a cover and barrier over the suture lines and the incisions.

12. The method of claim 10, wherein the sport injury comprises one or more selected from the group consisting of ACL injuries, Achilles tendon ruptures, frozen shoulder (adhesive capsulitis), ankle fractures and injuries, anterior and posterior cruciate ligament injury, chronic blister treatment, bursitis, carpal tunnel, cartilage injuries, elbow injuries, finger fractures, fractured clavicles, golfer's elbow (medial epicondylitis), torn groin muscle, hamstring tears, hand ischemia, hip pointer, patellofemoral pain syndrome, lateral epicondylitis (tennis elbow), medial and lateral collateral ligament injury, plantar fasciitis, quadricep pulls and tears, rotator cuff tears, shoulder tendinitis and impingement.

13. The method of claim 10, wherein the construct further comprises a frame that is disposable or implantable and resorbable.

14. The method of claim 10, wherein the construct has a shape selected from the group consisting of double wing shapes, single wing shapes, burn cover shapes, diamond shapes, articulating cup shapes, ligament, tendon and nerve tube shapes.

15. The method of claim 10, wherein the construct has one or more rounded or flattened corners.

16. The method of claim 10, wherein the construct comprises at least one layer of human amnion and at least one layer of chorion.

17. The method of claim 10, further comprising administering to the subject one or more additional treatments to the sport injury.

18. The method of claim 10, wherein the amniotic fluid and the human amnion and chorion tissues are obtained using a process comprising:
a. obtaining informed consent from pregnant females;
b. conducting risk assessment on the consented pregnant females to select an amnion donor;
c. procuring the amniotic fluid from the amnion donor;
d. procuring after birth placenta from the amnion donor; and
e. obtaining the human amnion and chorion tissues from the placenta.

19. A kit comprising:
a. at least one of an amniotic fluid and a construct for use in surgical repair of a sport injury; and
b. instructions on how to use the amniotic fluid and the construct in the surgical repair,
wherein the construct comprises an allograft comprising at least one layer of human amnion and chorion tissues, the construct has a size and shape suitable for covering a damaged site of fascia.

20. The kit of claim 19, comprising a plurality of constructs for use in surgical repair of a sport injury, wherein at least two of the plurality of constructs have different shapes or sizes.
